(19) United States
(12) Patent Application Publication
(10) Pub. No.: US 2022/0104830 A1
(43) Pub. Date: Apr. 7, 2022

(54) OCCLUSION MEANS, SYSTEM COMPRISING AN OCCLUSION MEANS AND AN INSERTION CATHETER, AND METHOD FOR PRODUCING THE SYSTEM

(71) Applicant: QATNA MEDICAL GMBH, Hechingen (DE)

(72) Inventor: Marcos Centola, Hechingen-Stein (DE)

(73) Assignee: QATNA MEDICAL GMBH, Hechingen (DE)

(21) Appl. No.: 17/421,868

(22) PCT Filed: Jan. 9, 2020
(86) PCT No.: PCT/EP2020/050433

§ 371 Date: Jul. 9, 2021

(30) Foreign Application Priority Data
Jan. 10, 2019 (DE) 10 2019 100 530.2

(51) Int. Cl. A61B 17/12

(52) U.S. Cl. CPC A61B 17/12177; A61B 17/12172; A61B 17/12122

(57) ABSTRACT

The invention relates to an occluder for closing the left atrial appendage of a patient, a system having a catheter and an occluder, and a method for providing the system, the occluder having a self-expandable frame, wherein a biological tissue is arranged on the frame on the outside thereof, which tissue covers said frame at least in part, wherein the biological tissue is stabilized by treatment by means of a reticulation method and, in order to make it durable, is wetted with a liquid medium or dried.

The invention relates to an occluder for closing the left atrial appendage (auricula cordis sinistra) of a patient. The invention also relates to an associated system for inserting an occluder into a patient and a method for providing the system.

The auriculae atrii or atrial appendages are protuberances of the atria of the heart in mammals. The left atrial appendage (LAA), known medically as the auricula cordis sinistra, is located next to the pulmonary artery tract and is a frequent site for blood clots that can lead to a stroke, especially in patients with atrial fibrillation. Preventing thrombi in the left atrial appendage is therefore an effective stroke prophylaxis in vulnerable patients.

For this stroke prophylaxis, implants have been developed that are introduced into the protuberances and close the access, for example using a Teflon film. These implants are referred to as LAA (left atrial appendage) occluders. These implants are inserted into the protuberances and anchored there in particular by means of anchoring elements, so that, in particular via the proximal end region thereof, they close off the access into the protuberances in a fluid-tight manner. Insertion mostly takes place using endovascular techniques, i.e. in particular by means of an insertion catheter through which the implant is brought to the place of use. The occluders are brought to the place of use in particular in a reduced-volume form and expanded there. Self-expanding materials are generally used for the occluders, for example shape-memory alloys. An occluder of this kind is previously known from WO 2015/079023 A1.

DE 10 2015 104 785 A1 also discloses a device for closing the protuberances by means of a closure element, wherein two anchoring elements fix the closure element without damaging any tissue.

In addition, US 2018/0338832 A1 and WO 2013/158608 A1 disclose devices for being arranged, inter alia, on the mitral valve by means of a minimally invasive procedure in which said mitral valve is replaced by an artificial valve or its function is improved.

In addition, EP 1 729 682 B1 discloses an endoluminal vascular prosthesis which is introduced into the aorta as a stent and which, when expanded, repairs an aneurysm (sacculation).

US 2011/184439 A1 also discloses an occluder for closing a hole in a vascular wall on both sides, wherein the occluder comprises two screens provided with a biological material, and a cylindrical central portion connecting the two screens. A further occluder for closing a hole in a vascular wall is disclosed in WO2006/036837A2.

The problem addressed by the present invention is in particular that of improving the biocompatibility of the known occluders.

This problem is solved by an occluder having the features of claim 1. Accordingly, an occluder, in particular for closing the left atrial appendage of a patient, is proposed. The occluder comprises a self-expandable frame, wherein a biological tissue is arranged on the frame, which tissue covers said frame at least in part. The biological tissue is arranged in particular on an outside of the frame. Biological tissue is used in particular to increase the biocompatibility of the occluder. The biological tissue can in particular be arranged on the outside of the frame and thus be in contact with the patient's natural tissue and/or with the patient's circulating blood. In this respect, the biological tissue can in particular be used to close the left atrial appendage in a fluid-tight manner when the occluder is in the arranged state. It is also conceivable that, after the occluder has been arranged in the left atrial appendage, a natural cell or skin layer is formed on the biological tissue and thus grows over the biological tissue. In particular, by providing the biological tissue, increased biocompatibility can be achieved. In particular, the biological tissue can be used instead of a Teflon covering, as disclosed in WO 2015/079023 A1, or a covering made of any other synthetic material. As a result, the biocompatibility of the occluder can be improved as a whole.

The biological tissue is stabilized in particular by treatment by means of a reticulation method. The reticulation can preferably take place by means of the crosslinking process using, in particular, glutaraldehyde and/or alcohol. For sterilization and/or in order to make it durable, the biological tissue can also be permanently wetted with a liquid medium or wetted and then dried. It is conceivable that the biological tissue is first sterilized with a liquid medium and then dried, in particular vacuum-dried. The liquid medium is preferably glycerol and/or a mono- to trihydric alcohol.

The frame can in particular consist of a flexible, self-expanding material or comprise a material of this kind. This material can be a metal or a plastics material. However, it is advantageously a metal alloy that has shape-memory characteristics. Nickel-titanium alloys, for example a nitinol alloy, are particularly preferred. The processes of manufacturing implants from these materials and reshaping by means of tempering have been described in many cases. Shape-memory metals of this type can be deformed under external pressure and, when this pressure is eliminated, they automatically reassume an impressed shape. This allows the occluder to be deformed into an insertion position in order to bring said occluder into its intended position by means of a catheter and then release the occluder, which expands and thereby assumes its impressed shape or pushes into the shape impressed thereon.

In an advantageous development of the invention, the biological tissue is animal tissue, in particular tissue of a mammal. A tissue of this kind is particularly suitable for being arranged on a frame of an occluder. In particular, a tissue of this kind has a comparatively high biocompatibility with the tissue of a human patient.

In a further advantageous embodiment of the invention, the tissue is a pericardium membrane. A membrane of this kind has proven to be particularly advantageous in terms of biocompatibility and for reliably closing the left atrial appendage in a fluid-tight manner. However, any other animal tissue in membrane format, such as peritoneum tissue, diaphragmatic tissue or submucosal tissue of the small intestine or pericardial tissue, would also be conceivable.

It is also advantageous for the occluder to have a substantially spherical outer contour in the expanded state. A spherical or ball-shaped outer contour of this kind allows the occluder to be particularly advantageously positioned in the left atrial appendage in order to close it. This is in particular regardless of how precisely the occluder or the insertion catheter for arranging the occluder is arranged relative to the left atrial appendage in the patient during release and in particular regardless of the size and contour of the left atrial appendage. Consequently, in this case, the self-expandable occluder is in particular impressed with such a shape that, in the expanded configuration, it develops into a spherical or substantially spherical outer contour and in so doing comes into contact with the inner walls of the left atrial appendage as the occluder is released. The occluder can come to rest, for example, along a circular line on the inside of the left atrial appendage.

It is particularly preferred that, when the occluder is in the arranged state, the biological tissue covers at least part, in particular at least half, more in particular at least two thirds, more in particular completely or substantially completely covers, the proximal hemisphere of the outside of the frame. As a result, the left atrial appendage can be closed by means of the biological tissue, in particular in order to reduce the risk of thrombus formation in the left atrial appendage and the resulting risk of stroke. It is conceivable that, in the arranged state, the occluder rests against the left atrial appendage along a circular line. Proceeding therefrom in the proximal direction, it is particularly conceivable that the outside of the frame is completely or substantially covered by biological tissue, so that only this is exposed to the blood circulation through the aorta. It is also conceivable that, after the occluder has been arranged, a skin layer is formed on the biological tissue by the patient's body, so that ultimately a newly formed skin layer is formed on the biological tissue and the left atrial appendage is thus closed.

It is particularly preferred for the biological tissue to be sewn to the frame, in particular by means of PTFE threads. Fastening using other fastening methods would also be conceivable. However, it is particularly easy to sew to the frame structure. Surgical seams, in particular PTFE seams, are suitable for this purpose.

It is also particularly preferred if, in order to insert the occluder into a patient, the frame can be transferred into an insertion position in which the occluder has a substantially tubular outer contour. In this state, the occluder therefore in particular has a reduced outer diameter and can thus be brought to the intended position, in particular by means of an insertion catheter. In particular, starting from a shape impressed on the occluder, this can therefore be converted into a compressed configuration and, after being released from the insertion catheter, develops into the self-expanded configuration impressed thereon and thereby come to rest against the inside of the left atrial appendage.

At least one X-ray marker, in particular a plurality of X-ray markers, is/are advantageously arranged on the frame. This allows the occluder to be positioned on the auricula cordis sinistra in a particularly precise manner, and for the occluder to be subsequently released. The X-ray markers can in particular be arranged at a uniform distance along the outside of the occluder. In particular, these can be arranged along a circumference of the in particular spherical occluder. The region in which the X-ray markers are arranged can come to rest against the inner wall of the left atrial appendage when the occluder is in the arranged state. The X-ray markers can therefore in particular be cut from a cutting plane which, when the occluder is in the arranged state, separates the proximal hemisphere from the distal hemisphere. It is conceivable for the biological tissue to also cover the X-ray markers.

The frame preferably comprises a tubular end portion at its proximal end. In the arranged state of the occluder, the tubular end portion is consequently in the region of the proximal end of the frame. In particular, an insertion catheter for arranging the occluder on the left atrial appendage can be inserted into the frame through this tubular end portion. It is conceivable for the biological tissue to have an opening, so that the insertion catheter can be inserted into the frame through the opening and then through the tubular end portion. The biological tissue can be designed to be elastically yielding such that, after the insertion catheter has been removed from the occluder, the opening in the tissue is closed in a fluid-tight or substantially fluid-tight manner, and thus finally the left atrial appendage can be closed in a fluid-tight or substantially fluid-tight manner.

In this context, in one particularly preferred development of the invention, the frame comprises a pot-shaped end portion at its distal end. This pot-shaped end portion can accordingly have a tubular frame portion and an adjoining base portion, in order to thus form a pot-shaped distal end portion in the region of the distal end of the occluder when in the arranged state on the atrial appendage. A portion of an insertion catheter can be arranged in or on this cup-shaped end portion, in order to bring the occluder to its intended position on the left atrial appendage.

In this context, in a particularly preferred development of the invention, the frame comprises, between the tubular end portion and the pot-shaped end portion, a plurality of branching and reconverging webs in order to form a net-like frame portion. A net-like or frame-like frame portion can therefore be provided between the proximal and distal end portions of the occluder. In the expanded configuration, this frame portion can assume a spherical shape or a substantially spherical shape.

It is particularly preferred if the frame is formed as one piece. The frame can therefore in particular comprise a distal and a proximal end portion as well as a net-like frame portion therebetween. The one-piece frame can be produced by means of laser cutting, by means of a water-jet method or by means of electrical discharge machining (EDM). In particular, the plurality of branching and reconverging webs can therefore be “cut out” from a tubular portion. However, it would also be conceivable for the net-like frame structure to be made from a braided structure.

The proximal hemisphere of the occluder advantageously has first anchoring means. Additionally or alternatively, the distal hemisphere has second anchoring means.

In the arranged state of the occluder, the proximal hemisphere can consequently have first anchoring means. These can in particular be designed as hooks and have a curved structure that has an end portion pointing in the proximal direction.

Additionally or alternatively, the distal hemisphere can have second anchoring means. These can in particular be designed as barbs and in particular comprise a rod-shaped portion which points in the proximal direction.

The biological tissue is preferably stabilized by treatment by means of a reticulation method prior to being arranged on the frame. It is therefore conceivable for the biological tissue to first be stabilized by means of a crosslinking process, for example using glutaraldehyde and/or alcohol. It is conceivable for the biological tissue, in particular after it has been fastened to the occluder, to then be stored in a liquid medium until the occluder is intended to be used, and to only be arranged on the catheter together with the occluder immediately prior to being arranged in the patient. However, it would also be conceivable in particular for the biological tissue to be dried and made durable in this manner. It is conceivable that this is first treated with glycerol and a mono- to trihydric alcohol. The tissue can then be dried and in particular vacuum-dried. Any other tissue drying process for making the biological tissue durable would also be conceivable. A tissue dried in this way can consequently be arranged on the frame of the occluder. In this state, the occluder can then be arranged in or on the insertion catheter. The system consisting of the insertion catheter and occluder, comprising the biological tissue, can then be sterilized by means of in particular gaseous ethylene oxide (EtO) and durably stored in this configuration for the duration of the shelf life.

The problem stated at the outset is also solved by a system for inserting an occluder into a patient and for arranging the occluder on the left atrial appendage of the patient, the system comprising an insertion catheter and an occluder according to the invention arranged thereon, and a method for providing the system. By means of the system, the occluder can consequently be supplied to the left atrial appendage and then released there. It is particularly conceivable for the occluder to have a tubular proximal end portion through which the insertion catheter is inserted into the occluder. The insertion catheter can in particular have an outer tube and an inner tube. The outer tube can in this case end in front of the inner tube in the distal direction. The outer tube can be movement-coupled to the proximal end portion of the occluder, while the inner tube can be movement-coupled to the distal end portion of the occluder. The distal end region of the inner tube can be inserted into the distal pot-shaped end portion of the occluder. It is conceivable that the inner tube and the outer tube can be displaced relative to one another in order to initially transfer the occluder into a stretched state, i.e. an insertion position, in which said occluder can in particular have a tubular configuration, and then bring the occluder to its intended location on the left atrial appendage. The occluder can then be released and push into its, in particular spherical, self-expanded configuration and thereby come to rest against the left atrial appendage.

It is also advantageous if, after the biological tissue has been applied to the frame, the occluder is already arranged within the insertion catheter for storage and/or for transport to the operator (pre-loaded system). As a result of sterilizing the system consisting of the insertion catheter and the occluder by means of in particular ethylene oxide (ETO), the system can be durably stored and transported. This is also advantageous immediately before the system is introduced into the patient, since the occluder is not provided as a separate part from the insertion catheter and the operator therefore does not have to arrange the occluder on the insertion catheter. This embodiment can therefore save a work step and there is consequently less risk of contamination or operating errors.

In the method for providing a system according to the invention, biological tissue is first stabilized by means of a reticulation method, and then the biological tissue is wetted with a liquid medium and/or dried for sterilization and in order to make it durable. The biological tissue can be dried, in particular by means of vacuum drying, and can then be arranged on the occluder and introduced into the insertion tube (dry state). The entire system, together with the occluder present in the insertion tube, can then be made available to the operator, in particular the doctor (pre-loaded system). However, it is also conceivable for the occluder comprising the biological tissue to be made available to the operator separately from the insertion catheter in a sterilizing liquid bath (wet state), and for the occluder to be fastened to the insertion catheter immediately before the system is used (user-loaded system).

Further details and advantageous embodiments of the invention can be found in the following description, on the basis of which the embodiment of the invention shown in the figures is described and explained in more detail.

In the drawings:

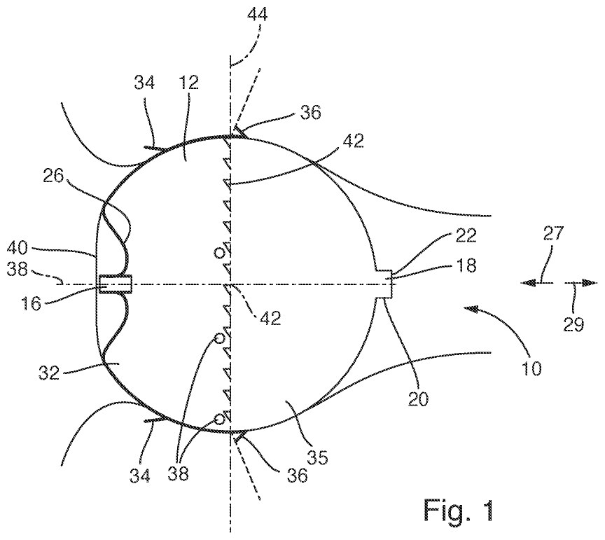

FIG. 1 is a schematic plan view of an occluder according to an embodiment that is arranged on the left atrial appendage;

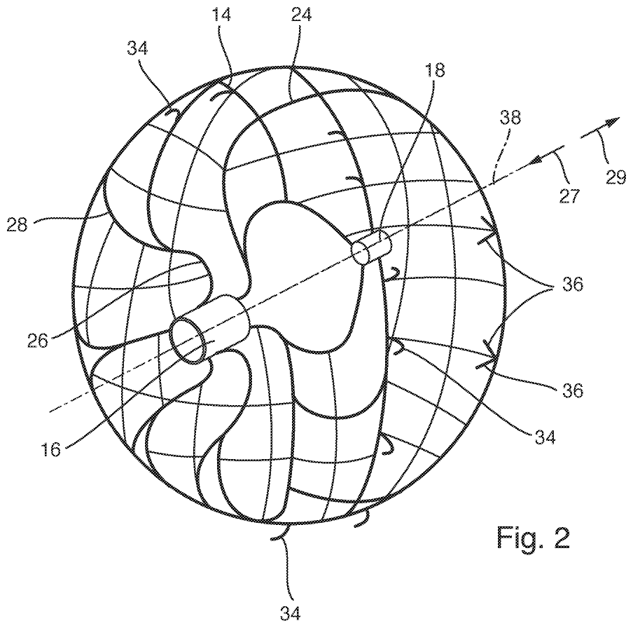

FIG. 2 is a schematic perspective view of the occluder according to FIG. 1;

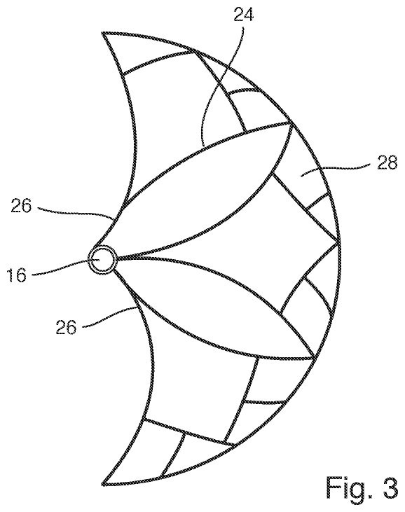

FIG. 3 is a plan view of one half of the occluder according to FIG. 2;

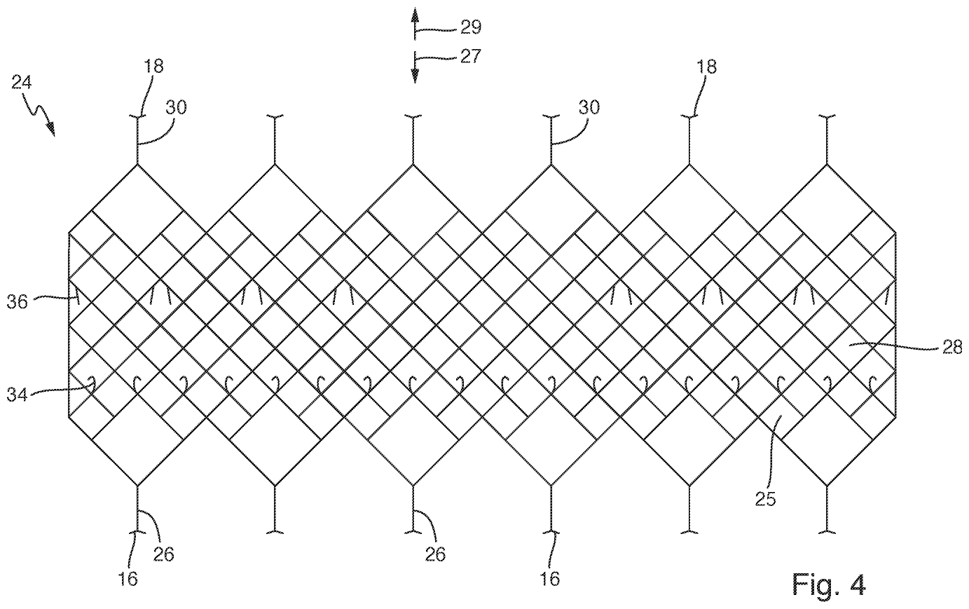

FIG. 4 is a cutaway schematic view of a frame portion of the occluder according to FIG. 2;

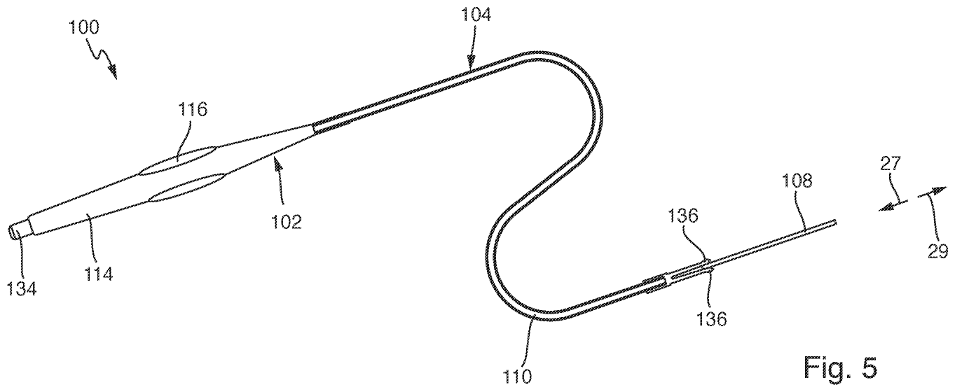

FIG. 5 is a perspective schematic view of an insertion unit according to an embodiment;

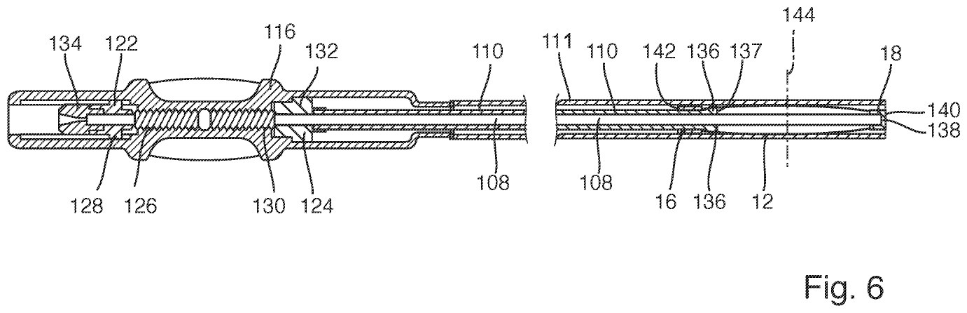

FIG. 6 is a schematic cross-sectional view of the insertion unit according to FIG. 1, with an occluder arranged in an insertion tube in the insertion position;

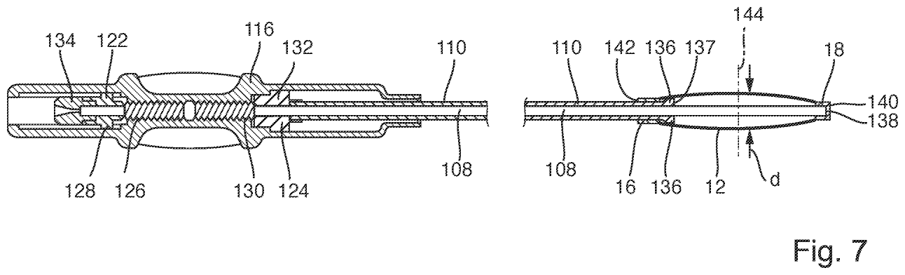

FIG. 7 is a schematic cross-sectional view corresponding to FIG. 6, without an insertion tube;

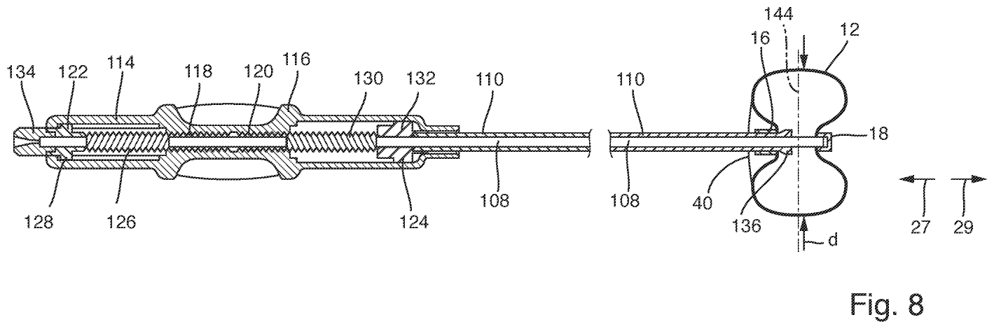

FIG. 8 is a schematic cross section of the insertion unit according to FIG. 1 with the occluder arranged thereon, in a configuration before the insertion unit is removed from the occluder.

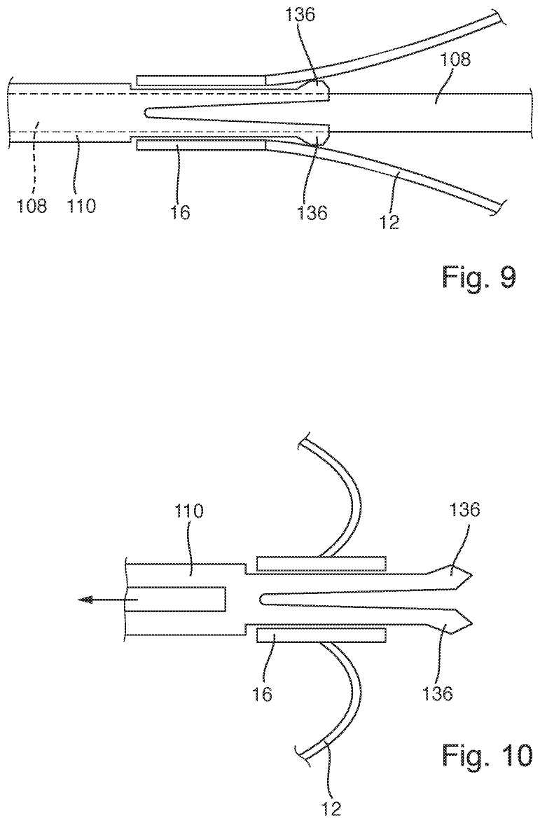

FIG. 9 is a schematic cross section of a region around the proximal end of the occluder, with the insertion unit arranged thereon;

FIG. 10 is a view based on FIG. 9, with the inner tube of the insertion unit removed from the occluder;

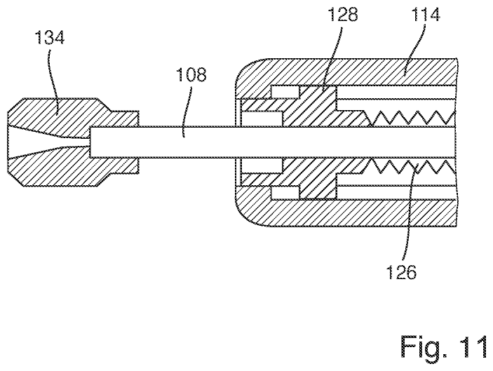

FIG. 11 is a view of a proximal region of the insertion unit with a Luer connector detached from the insertion unit; and

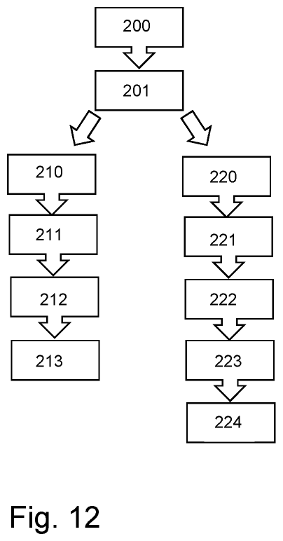

FIG. 12 shows method steps for providing a system according to the invention.

Firstly, FIG. 1 schematically shows the left atrial appendage of a patient, denoted with reference sign 10. To reduce the risk of a stroke, an occluder 12 is introduced into the left atrial appendage 10 in order to close the access to the left atrial appendage 10.

The occluder 12 initially comprises a frame 14 formed as one piece. This frame comprises a proximal tubular portion 16 and a pot-shaped distal end portion 18. The pot-shaped distal end portion 18 comprises a circular-cylindrical sheath portion 20 and a base portion 22, in order to form a pot-shaped structure. The occluder has a net-like frame portion 24 between the two end portions 16, 18. This net-like frame portion 24 can be seen particularly clearly in a cut-open form in FIG. 4. Starting from the proximal tubular end portion 16, the net-like frame portion 24 initially has a number of webs 26. These merge into a branching network of webs 25 in order to form the net structure. In so doing, diamond-shaped structures 28 are formed. In the distal direction 29, the webs 25 converge again into individual webs 30 which open into the distal end portion 18.

In the arranged state (cf. FIG. 1), the occluder 12 has a proximal hemisphere 32 and a distal hemisphere 35. A number of first anchoring means 34 are provided in the region of the proximal hemisphere 32. These extend along a circular line along the circumference and are in the form of hooks having end portions pointing in the proximal direction 27. Second anchoring means 36 are formed in the region of the distal hemisphere 35. These likewise extend along a circular line along the circumference, have a rod-like shape and protrude obliquely from the circumferential surface in the proximal direction 27. The anchoring means 34, 36 are also formed as one piece with the frame 14.

The frame 14 of the occluder 12 consists of a self-expanding material, for example a shape-memory alloy, in particular a nitinol alloy. The expanded shape impressed on the occluder 12 is spherical (cf. FIG. 2). The occluder 12 has a longitudinal axis 38 extending in the proximal or distal direction through its center point. This longitudinal axis 38 also extends through the central longitudinal axis of the tubular proximal end portion 16 and through the central longitudinal axis of the distal end portion 18 (cf. FIG. 2). The proximal hemisphere 32 is completely covered by a biological tissue 40. This biological tissue 40 is in particular a biological membrane. In particular, it can be the pericardium membrane. The tissue 40 comprises openings, such that the first anchoring means 34 protrude through the openings. The tissue 40 also has an insertion opening in order to insert an insertion catheter through the proximal tube portion 16 into the occluder 12. When the insertion catheter has been removed from the occluder 12 after the occluder 12 has been released, the elastically yielding tissue 40 can contract in such a way that the insertion opening is closed in a substantially fluid-tight manner, so that overall the tissue 40 closes off the proximal hemisphere 32 in a substantially fluid-tight manner and substantially covers the frame 14. As indicated schematically by surgical threads 42, the tissue is sewn to the frame 14 by means of PTFE threads.

In the vicinity of the parting plane 44 of the proximal hemisphere 32 and the distal hemisphere 35, a number of X-ray markers 38 are placed over the circumference in the region of the proximal hemisphere 32. These allow the occluder 12 to be precisely positioned in the left atrial appendage. A surgeon can consequently place the occluder 12 in a particularly precisely positioned manner.

Overall, by providing the biological tissue 40, an occluder 12 that has comparatively high biocompatibility can be provided. In this case, after the occluder 12 has been arranged on the left atrial appendage, the patient's natural tissue can grow over the biological tissue 40. As a result of the biological tissue 40 used, there is overall high biocompatibility and thus an increased probability that surgical intervention to close the left atrial appendage is successful.

In the following, a system for inserting the occluder 12 into a patient and for releasing the occluder 12 in the left atrial appendage 10 of the patient is described according to one embodiment:

FIG. 5 shows an insertion unit 100 as a whole. This comprises both a drive unit 102 and an insertion catheter 104. The insertion catheter 104 comprises an inner tube 108 and an outer tube 110. The inner tube 108 extends through the outer tube 110. Moreover, the outer tube 110 ends in front of the inner tube 108 in the distal direction 29. As shown in FIG. 6, the insertion catheter 104 can also have an insertion tube 111 surrounding the outer tube 110. The drive unit 102 comprises a housing 114 which can be held in the hand of an operator, in particular a surgeon, and which overall has an elongate shape.

The drive unit 102 also comprises an actuating element 116 which is rotatably arranged in the housing 114. The actuating element 116 is hollow and has a first drive thread 118 and a second drive thread 120, the first drive thread 118 being proximal to the second drive thread 120 (cf. FIGS. 6, 7 and 8). The actuating element 116 is in particular formed as one piece.

A first transmission element 122 and a second transmission element 124 are also arranged in the housing 114. The two transmission elements 122, 124 overall have a helical shape. The first transmission element 122 has a thread portion 126 and a head portion 128. The second transmission element 124 correspondingly has a thread portion 130 and a head portion 132. The two transmission elements 122, 124 can in particular each be formed as one piece. The first transmission element 122 is in this case movement-coupled to the inner tube 108, while the second transmission element 124 is movement-coupled to the outer tube 110. The head portion 128 of the first transmission element 122 interacts with a Luer connector 134 that is fastened to the inner tube 108. The Luer connector 134 is detachably arranged on the head portion 128 of the first transmission element 122.

The head portion 132 of the second transmission element 124 interacts with the outer tube 110 for the purpose of movement coupling. The outer tube 110 is detachably arranged on the head portion 132.

At the distal end of the outer tube 110, said tube has two latching finger-like end portions 136, which can be seen particularly clearly in FIG. 10. These are formed as one piece with the outer tube 110 and can be deformed in an elastically yielding manner. Overall, the mode of operation of the insertion unit 100 is then as follows:

In order to insert the occluder 12 into the left atrial appendage 10, the insertion catheter 104 is firstly arranged on the occluder 12 in order to form a system consisting of the insertion unit 100 and occluder 12, in order to bring the occluder 12 to the left atrial appendage 10 and then release the occluder 12.

The occluder 12 is initially in its self-expanded shape and thus has a spherical outer contour (cf. FIG. 2). The outer tube 110 is inserted into the occluder 12 through an insertion opening (not shown) in the biological tissue 40 of the occluder 12 and through the tubular proximal tube portion 16. The inner tube 108 is then passed through the occluder 12 and the outer tube 110 and arranged on the distal pot-shaped end portion 18 of the occluder 12. The latching finger-like portions 136 are secured against elastic deformation in the radially inward direction by the inner tube 108, so that the latching fingers 136 come to rest against the proximal tubular portion 16 in a form-fitting manner.

The housing 114 can then be picked up by an operator, in particular a surgeon, and the actuating element 116 can be rotated. As shown in FIG. 7, the actuating element 116 is first rotated in such a way that the head-like portions 128, 132 of the two transmission elements 122, 124 are moved toward one another. As a result, the distal end 137 of the outer tube 110 and the distal end 138 of the inner tube 108 are moved away from one another. As a result, the distal end 140 of the occluder 12 is displaced in the distal direction 29, while the proximal end 142 of the occluder is displaced in the proximal direction 27. Overall, the occluder 12 is in this case transferred into an insertion position, so that the occluder 12 as a whole assumes a compressed form in which it has, by comparison with the self-expanded form, an elongate outer contour having a reduced diameter d (cf. FIG. 7). The occluder is therefore brought into its insertion position by the operator (user-loaded system).

Alternatively, it is conceivable for the occluder 12 to be made available on the insertion catheter 104 as a preconfigured system; the occluder 12 can be surrounded and protected by the insertion tube 111 and is made available “loaded” in its insertion position (pre-loaded system). The occluder 12 can in particular comprise the biological tissue that has been made durable, in the insertion tube 111. The biological tissue can be conditioned using a liquid medium, preferably in an alcohol such as glycerol, and then be vacuum-dried. Furthermore, the biological tissue can be sterilized by means of ethylene oxide (EtO) and, in this configuration, can be durably stored in the insertion catheter 104 or the insertion tube 111 thereof in the compressed insertion position.

In the compressed insertion position, as described above, the occluder 12 can be inserted into a blood vessel together with the insertion catheter 104 and then brought further up to the left atrial appendage to the intended position. The position of the occluder 12 can be determined by means of the X-ray markers 38. The central region of the occluder 12, i.e. the region of the X-ray markers 38, is intended to come to rest against the left atrial appendage.

In the event that the insertion catheter 104 comprises the insertion tube 111, said tube is withdrawn with respect to the outer tube 110 when the occluder 12 has reached its position on the left atrial appendage 10, whereby the occluder can be released.

In order to fully release the occluder 12, the actuating element 116 is rotated, as shown in FIG. 8, such that the head-like portions 128, 132 are moved away from one another. As a result, the inner tube 108 is moved in the proximal direction 27, while the outer tube 110 is moved in the distal direction 29. In the course of this relative movement, the proximal end 142 of the occluder 12 and the distal end 140 of the occluder 12 are moved toward one another. As a result, the diameter d of the occluder 12 increases. The occluder 12 pushes into its self-expanded shape, so that the proximal end 142 and the distal end 140 of the occluder push toward one another.

In this case, the position of the central part of the occluder 12 remains in particular unchanged, which is shown in FIGS. 7 and 8 with reference to the center line 144. The center line 144 extends through the middle of the occluder 12 between the proximal end 142 and the distal end 140 when said occluder, as shown in FIG. 7, has reached the intended position. The middle of the occluder 12 therefore remains in the correct position and thus does not change position when the occluder is released, as shown in FIG. 8.

In the position shown in FIG. 7, the inner tube 108 is held on the pot-shaped distal end portion 18 of the occluder 12 in a force-fitting manner in the proximal direction 27. Consequently, during a proximal movement of the inner tube 108, the distal end portion 18 of the occluder 12 is also moved in the proximal direction 27. When the occluder 12 is transferred into a position as shown in FIG. 8, the force of the force fit is reduced so that, as shown in FIG. 11, the Luer connector 134 can be detached from the head-like portion 128 with comparatively little effort, so that the Luer connector 134 and thus the inner tube 108 can be pulled off the occluder 12 through the outer tube 110.

Then, as shown in FIG. 11, the outer tube 110 can be pulled off the occluder 12, since the latching finger-like portions 136 can now move elastically radially inward. The occluder 12 can then assume its release position in which it can assume its self-expanded shape, as shown in FIG. 1, or in any case pushes into its self-expanded final shape and thus tightly closes the left atrial appendage. When the insertion catheter has been removed from the occluder 12 after the occluder 12 has been released, the elastically yielding tissue 40 can contract in such a way that the insertion opening (not shown) in the tissue 40 is closed in a substantially fluid-tight manner, so that overall the tissue 40 closes off the proximal hemisphere 32 in a substantially or completely fluid-tight manner and substantially or completely covers the frame 14. The patient's skin can then grow over the biological tissue 40, so that the left atrial appendage can be permanently and stably closed.

As a result of the proposed configuration, the insertion catheter 104 can be pull off the occluder 12 in a particularly simple manner. This can be achieved in particular without, or almost without, a torque being exerted on the occluder 12. This reduces the risk of the occluder being displaced from its intended position in an undesirable manner when the insertion catheter 104 is pulled off the occluder 12.

The method for providing a system according to the invention is shown schematically in FIG. 12 and provides the following steps:

In method step 200, biological material, in particular pericardium, is first provided, and in method step 201 it is stabilized by a reticulation method (crosslinking), in particular by means of glutaraldehyde and/or alcohol.

In a next method step 210, the reticulated biological material can then be fastened to the occluder or to its frame and in particular be sewn on. In the subsequent method step 211, the occluder comprising the biological material is preserved in a liquid medium and thus made durable. The liquid medium can be glutaraldehyde and/or an alcohol. In method step 212, the insertion catheter is sterilized independently of the occluder, in particular using gaseous ethylene oxide (EtO). The occluder and the insertion catheter can then be packaged separately from one another and made available to the doctor. In method step 213, generally in the operating theater, the wetted occluder is then mounted on or arranged in the insertion catheter (user-loaded system).

Instead of method steps 210 to 213, method steps 220 to 224 can follow method step 201. In method step 220, the biological material is dried in particular by means of glycerol and/or a mono- to trihydric alcohol, specifically in particular vacuum-dried or air-dried. In method step 221, the biological material is fastened, in particular sewn, to the occluder or the frame thereof. In method step 222, the occluder is introduced, together with the biological material, into the insertion catheter. In method step 222, the occluder comprising the biological material is sterilized inside the insertion catheter, in particular using gaseous ethylene oxide (EtO). The system pre-assembled in this way can then be packaged and stored accordingly and made available to the doctor in the operating theater as a pre-loaded system in method step 224. The system is then ready to use.

What is claimed is:

1. Occluder for closing the left atrial appendage of a patient, comprising a proximal end and a distal end, and comprising a self-expandable frame,
characterized in that, in the expanded state, the frame has a substantially spherical outer contour,
in that, in an insertion position, the occluder has, by comparison with the self-expanded form, an elongate outer contour in which the proximal end is displaced in the proximal direction and the distal end is displaced in the distal direction,
in that on the outside of the proximal hemisphere of the frame, at least partially covering biological tissue is arranged and
in that the biological tissue is stabilized by treatment by means of a reticulation method and, in order to make it durable, is either wetted with a liquid medium or dried.

2. Occluder according to claim 1, characterized in that the reticulation method comprises the crosslinking process using glutaraldehyde and/or alcohol.

3. Occluder according to claim 1, characterized in that the liquid medium with which the biological tissue is wetted is glutaraldehyde and/or alcohol.

4. Occluder according to claim 1, characterized in that the biological tissue is initially wetted with glycerol and/or with a mono- to trihydric alcohol and is then dried.

5. Occluder according to claim 4, characterized in that the biological tissue is vacuum-dried or air-dried.

6. Occluder according to claim 5, characterized in that the dried biological tissue is sterilized with ethylene oxide.

7. (canceled)

8. Occluder according to claim 1, wherein, in order to insert the occluder (12) into a patient, the frame can be transferred into an insertion position in which the occluder has a substantially tubular outer contour.

9. Occluder according to claim 1, wherein the frame comprises a tubular end portion at its proximal end and/or wherein the frame comprises a pot-shaped end portion at its distal end.

10. Occluder according to claim 1, wherein the proximal hemisphere has first anchoring means, and/or wherein the distal hemisphere (35) has second anchoring means.

11. System for inserting an occluder into a patient and for arranging the occluder on the left atrial appendage (10) of the patient, the system comprising an insertion catheter and occluder comprising biological tissue.

12. System according to claim 11, characterized in that the occluder is arranged in an insertion position in a compressed configuration within the insertion catheter, and in that the arrangement is such that the occluder can be supplied to the left atrial appendage in the insertion position and released there.

13. System according to claim 12, characterized in that the insertion catheter comprises an inner tube, an outer tube and an insertion tube, the occluder comprising the biological tissue being accommodated in the insertion tube.

14. Method for providing a system for inserting an occluder into a patient and for arranging the occluder on the left atrial appendage of the patient, the system comprising an insertion catheter and occlude comprising biological tissue, wherein the biological tissue is first stabilized by means of a reticulation method and, in order to make it durable, is either wetted with a liquid medium, or dried.

15. Method according to claim 14, wherein the liquid medium comprises glutaraldehyde and/or alcohol and the occluder comprising the biological tissue is arranged on the insertion catheter immediately before it is inserted into the patient.

16. Method according to claim 14, wherein the biological tissue is vacuum-dried or air-dried, and wherein the occluder comprising the biological tissue is introduced into the insertion catheter and, having been made durable, is made available.

17. Occluder for closing the left atrial appendage of a patient, comprising a self-expandable frame, characterized in that biological tissue at least partially covering the frame is arranged on the outside of the frame, in that the occluder has a substantially spherical outer contour in the expanded state, in that the occluder comes to contact the inner side of the auricle cordis sinistra along a circular line, and in that the frame is formed in one piece.
